SEQUENCE LISTING (2 sequences)

SEQ ID NO: 1 - DNA, 3898 bp, Artificial Sequence
  pFVS Vector
ccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagcgctttttagctttaaaccgcggggatcccaaacttcttctggaggtaccgcatgcgatttcgagctctcccggcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcaga

SEQ ID NO: 2 - DNA, 596 bp, Artificial Sequence
  Synthetic DNA
gccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagcgctttttagctttaaaccgcggggatcccaaacttcttctggaggtaccgcatgcgatttcgagctctcccggcaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggt

(19) United States
(12) Patent Application Publication
(10) Pub. No.: US 2020/0277612 A1
(43) Pub. Date: Sep. 3, 2020

(54) VECTORS FOR VARIABLE REGION SEQUENCE SCREENING

(71) Applicant: BRISTOL-MYERS SQUIBB COMPANY, Princeton, NJ (US)

(72) Inventor: Xiang SHAO, Milpitas, CA (US)

(21) Appl. No.: 16/753,102

(22) PCT Filed: Oct. 2, 2018
(86) PCT No.: PCT/US2018/053890

§ 371 Date: Apr. 2, 2020

Related U.S. Application Data
(60) Provisional application No. 62/567,421, filed on Oct. 3, 2017.

(51) Int. Cl. C12N 15/63; C07K 16/00; G01N 33/53

(52) U.S. Cl. CPC C12N 15/63; G01N 33/53; C07K 16/00

(57) ABSTRACT

Described herein are vectors and methods that are useful for screening variable region sequences of antigen binding molecules with high efficiency. Such vectors rely on the native translation initiation sequences of the variable regions to drive the expression of both the variable region and a reporter gene in the vector.

### CROSS REFERENCE TO RELATED APPLICATION

This application claims the benefit of U.S. Provisional Application Ser. No. 62/567,421 filed on Oct. 3, 2017, which is hereby incorporated by reference in its entirety.

### SEQUENCE LISTING

This application includes as part of its disclosure a biological sequence listing in a file named “12976WOPCT_Sequences_ST25.txt” and having a size of approximately 7 KB, created on Sep. 13, 2018, the content of which is hereby incorporated by reference in its entirety.

### BACKGROUND

Recent developments in antibody technology and the successful application of antibodies as therapeutics have increased the demand for efficient antibody variable region sequencing methods. The sequencing of antibody variable regions (VH and VL) from hybridomas, clonal B cells, or combinatorial display hits is a critical step in numerous applications, including recombinant antibody production in various formats, antibody optimization, and database banking One step in the antibody variable region Sanger sequencing process that can benefit from improved efficiency is the initial vector-based screening of candidate sequences to differentiate variable regions of a target antibody from those derived from, e.g., pseudogenes, mRNAs encoding non-functional antibodies, and non-specific sequences. Major contributors to reduced efficiency in antibody variable region sequencing methods are false positives and false negatives. Accordingly, screening methods that reduce these inefficiencies would be highly beneficial to antibody drug development. Such methods can also be useful for screening and sequencing T cell receptor (TCR) variable regions.

### SUMMARY

Provided herein are a vector platform and methods for the efficient cloning, screening, sequencing, and identification of variable regions of antigen binding molecules such as antibodies and TCRs. The vectors and methods disclosed herein may be used for efficient cloning, screening and sequencing of any polypeptides.

In one aspect, provided herein is a vector comprising a cloning site for an antibody variable region (e.g., a heavy chain or light chain variable region) upstream of a reporter gene, wherein the reporter gene lacks a translation initiation sequence, and wherein the native translation initiation sequence of the antibody variable region drives the expression of the antibody variable region and reporter gene. In some embodiments, the vector comprises a nucleic acid encoding an antibody variable region.

In another aspect, provided herein is a vector comprising (i) a cloning site for directional cloning of a nucleic acid encoding an antigen binding molecule comprising a native translation initiation sequence, and (ii) a reporter gene lacking translation initiation sequence, wherein the cloning site is upstream of the reporter gene, and when the nucleic acid encoding the antigen binding molecule is cloned into the cloning site, the expression of the reporter gene is driven by the native translation initiation sequence in the nucleic acid encoding the antigen binding molecule.

In some embodiments, the vector comprises a promoter (e.g., prokaryotic promoter such as a lac promoter or a eukaryotic promoter) for expressing the antigen binding molecule, unique restriction sites (e.g., one or more restriction sites unique in the vector), a reporter gene (e.g., a gene encoding an enzyme, a chromogenic protein, a fluorescent protein, or a toxic gene), and/or a selectable marker gene (e.g., antibiotic resistance gene, a gene essential for growth of a host cell, or a gene required for replication and propagation of the vector in a host cell). In some embodiments, the antigen binding molecule and reporter gene are expressed as a fusion protein.

In one embodiment, the vector comprises the nucleotide sequence of SEQ ID NO: 1.

In another aspect, provided herein is a kit comprising a vector disclosed herein, and instructions for use. In one embodiment, the vector is linearized.

In another aspect, provided herein is a method of screening for variable regions (e.g., heavy or light chain variable region) of an antibody comprising:
a) amplifying one or more antibody variable regions using gene specific primers;
b) cloning the amplified product (e.g., by IN-FUSION cloning) into a vector described herein;
c) transforming the vector of step b) into a host cell (e.g., bacterial cell, mammalian cell, yeast cell); and
d) screening for cells that express the reporter gene (e.g., lacZα),

wherein high levels of expression of the reporter gene is indicative of the cloning of a full length variable region, and low levels or no expression of the reporter gene is indicative of the cloning of a non-full length variable region or no insert.

In another aspect, provided herein is a method of screening for antigen binding molecules comprising:
a) amplifying a nucleic acid encoding an antigen binding molecule using gene specific primers;
b) cloning the amplified nucleic acid into the vector disclosed herein, wherein the amplified nucleic acid is inserted in-frame with the reporter gene in the vector;
c) transforming the vector resulting from step b) into host cells; and
d) screening for cells that express the protein encoded by the reporter gene,

wherein expression of the protein encoded by the reporter gene is indicative of presence of a native translation initiation sequence in the amplified nucleic acid encoding the antigen binding molecule.

In another aspect, provided herein is a method of screening for antigen binding molecules comprising:
a) amplifying a nucleic acid encoding an antigen binding molecule using gene specific primers;
b) cloning the amplified nucleic acid into a vector, wherein the vector comprises

(i) a cloning site for directional cloning of a nucleic acid, and

(ii) a reporter gene lacking translation initiation sequence,

wherein the cloning site is upstream of the reporter gene, and wherein the amplified nucleic acid is inserted into the cloning site in-frame with the reporter gene;
c) transforming the vector containing the amplified nucleic acid into host cells; and
d) screening for cells that express the protein encoded by the reporter gene.

In some embodiments, expression of the protein encoded by the reporter gene is indicative of presence of a native translation initiation sequence in the amplified nucleic acid encoding the antigen binding molecule.

In some embodiments, the method further comprises the steps of selecting a cell or cells that express the reporter gene and sequencing the variable regions cloned upstream of the reporter gene.

### BRIEF DESCRIPTION OF THE DRAWINGS

FIG. 1 is a schematic of the pFVS vector.

FIG. 2 is a chart comparing features of the commercial pCR4.TOPO vector with the pFVS vector.

FIG. 3 is a bar graph showing variable region sequences obtained using the pCR4.TOPO vector (TA) compared to the pFVS vector.

### DETAILED DESCRIPTION

The present invention provides an improved vector platform and methods which rely on the native translation initiation elements of a nucleic acid encoding a polypeptide of interest to drive the expression of a reporter gene, thereby allowing for highly efficient screening of full length polypeptides. The vector substantially reduces the amount of false positives and false negatives in screening methods, and thus can be reliably used for direct, directional cloning of PCR-amplified DNA fragments.

In one aspect, provided herein is a vector comprising (i) a cloning site for directional cloning of a nucleic acid encoding a polypeptide comprising a native translation initiation sequence, and (ii) a reporter gene lacking translation initiation sequence, wherein the cloning site is upstream of the reporter gene, and when the nucleic acid encoding the polypeptide is cloned into the cloning site, the expression of the reporter gene is driven by the native translation initiation sequence in the nucleic acid encoding the polypeptide. In some embodiments, the polypeptide may be an antigen binding molecule. In some embodiments, the antigen binding molecule may be an antibody variable region, such as a heavy chain variable region, e.g., full length heavy chain variable region, or a light chain variable region, e.g., a full length light chain variable region. In some embodiments, the antigen binding molecule may be a variable region in a TCR.

Also provided are methods of screening a variable region repertoire for variable region discovery.

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked (also referred to as “expression vectors”). In general, expression vectors of utility in recombinant DNA techniques are often in the form of plasmids. In the present specification, “plasmid”, “vector” and “expression vectors” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “directional cloning” is used herein to refer to methods of directing the orientation of clonal inserts into a vector in a specific orientation. The term “native translation initiation sequence” is used herein to refer to the translation initiation sequence that naturally occurs in a nucleic acid encoding a polypeptide, such as an antigen binding molecule, e.g., an antibody or TCR variable region.

The term “antigen binding molecule” is used herein to refer to a polypeptide that may bind to an antigen. In some embodiments, the antigen binding molecule may be an antibody heavy chain or a fragment thereof. In some embodiments, the antigen binding molecule may be an antibody heavy chain variable region or a fragment thereof. In some embodiments, the antigen binding molecule may be an antibody light chain or a fragment thereof. In some embodiments, the antigen binding molecule may be an antibody light chain variable region or a fragment thereof. In some embodiments, the antigen binding molecule may be a TCR protein chain or a fragment thereof. In some embodiments, the antigen binding molecule may be a TCR alpha (α), beta (β), gamma (γ), or delta (δ) chain, or a fragment thereof. In some embodiments, the antigen binding molecule may be a variable region of TCR alpha (α), beta (β), gamma (γ), or delta (δ) chain, or a fragment thereof.

The term “antibody” refers, in some embodiments, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL).

The term “T cell receptor” or “TCR” refers, in some embodiments, to a protein found on the surface of T cells, or T lymphocytes, comprising two different protein chains inter-connected by disulfide bonds. TCR is responsible for recognizing fragments of antigen as peptides bound to major histocompatibility complex (MHC) molecules. In humans, the TCR may comprises an alpha (α) chain and a beta (β) chain, or a gamma and delta (γ/δ) chains.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein. In some embodiments, the host cell may be a bacterial cell, such as E. coli. In some embodiments, the host cell may be a mammalian cell, such as CHO cell or HEK cell. In some embodiments, the host cell may be a yeast cell.

The term “enzymatically active variant” used herein means a variant of an enzyme that retains at least some of its enzymatic activity.

The term “gene specific primers” as used herein refers to primers designed to amplify a nucleic acid encoding an antigen binding molecule or a fragment there of For example, a reverse (5′→3′ antisense strand) primer for PCR may be designed to anneal to a nucleotide sequence encoding a constant region of antigen-binding molecule, such as an antibody or TCR; the reverse primer, paired with a 5′ universal forward primer (5′→3′ sense strand), may be used to amplify the variable region of the antigen binding molecule, such as a full-length variable region of an antibody or TCR.

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand. For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

In some embodiments, the polypeptide of interest is an antibody variable region, for example, a heavy chain variable region or light chain variable region. Accordingly, in a vector comprising a nucleic acid encoding an antibody variable region upstream of the reporter gene, the native translation initiation sequence of the variable region drives expression of both the variable region and reporter gene (e.g., as a fusion protein). In some embodiments, the polypeptide of interest is an enzyme, Adnectin, or TCR variable region.

In some embodiments, the vector is a bacterial vector. In some embodiments, the vector is a mammalian vector. In some embodiments, the vector is a yeast vector. In some embodiments, the vector is a plant vector. In some embodiments, the vector is an insect vector.

In some embodiments, the vectors encode one or more reporter genes, wherein at least one of the reporter genes lacks a translation initiation sequence (e.g., ATG). Accordingly, the reporter gene is expressed only when a nucleic acid sequence encoding a polypeptide (e.g., an antibody or TCR variable region) is present upstream and in-frame with the reporter gene, such that the native translation initiation sequence of the nucleic acid encoding the polypeptide drives expression of both the polypeptide and the reporter gene (e.g., as a fusion protein). That is, without a nucleic acid encoding the polypeptide having a native translation initiation sequence upstream and in-frame with the reporter gene, the reporter gene is not expressed or expressed at very low levels. In some embodiments, the reporter gene encodes an enzyme, a chromogenic protein, a fluorescent protein, or a toxic gene. Exemplary reporter genes include, for example, GFP, YFP, RFP, EGFR, orange fluorescent protein, cyan fluorescent protein, substituted p-nitrophenyl phosphate, beta-galactosidase, luciferase, alkaline phosphatase, secreted alkaline phosphatase, beta-glucouronidase, and derivatives and variants thereof. The function of the reporter genes are easily assayed qualitatively or quantitatively using art-recognized methods. For example, when the reporter gene is a beta-galactosidase alpha fragment (lacZα) (including enzymatically active variant thereof), the lacZα fragment is transcribed and translated, and forms a functional beta-galactosidase enzyme. The substrate X-gal, when cleaved, leaves a water-insoluble blue product that marks the colonies. Therefore, the function of the gene can be assayed using IPTG and X-gal in blue-white screening (e.g., as described in Example 2). In another embodiment, when the reporter gene is GFP, the colonies fluoresce under blue or ultra violet light.

The vectors comprise a cloning site for a nucleic acid encoding a polypeptide (e.g., an antibody variable region) located upstream (5′) of the nucleic acid encoding the reporter gene. In some embodiments, the cloning site comprises multiple restriction enzyme sites. In order to allow for directional cloning (i.e., cloning in one orientation only), in some embodiments, the cloning site comprises at least one restriction site that is unique in the vector (i.e., present only once in the vector). In some embodiments, the cloning site comprises two restriction sites that are unique in the vector. An exemplary pair of restriction enzyme sites is AfeI and SacII. However, it will be understood by those of ordinary skill that any unique restriction enzyme site or sites can be used in the vector for cloning the nucleic acid encoding the polypeptide. In certain embodiments, recombinational cloning (e.g., the IN-FUSION® Cloning system) that uses in vitro site-specific recombination is used to accomplish the directional cloning of nucleic acids encoding polypeptides into the vectors described herein. In some embodiments, the vector disclosed herein comprises a nucleic acid encoding an antigen binding molecule comprising a native translation initiation sequence.

In some embodiments, the vectors comprise one or more selection markers, for example, an antibiotic resistance gene, that allow for contamination-free growth of the host cells harboring the vector. In some embodiments, the selection marker gene is a gene essential for growth of a host cell, or a gene required for replication and propagation of the vector in a host cell. Transformants that have such selection markers can be selected for by culturing in media containing the drug to which the gene is resistant (e.g., antibiotic). Exemplary antibiotic resistance genes suitable for use in the vectors described herein include zeocin resistant gene, a kanamycin resistant gene, a chloramphenicol resistant gene, a puromycin resistant gene, an ampicillin resistant gene, a URA3 gene, a hygromycin resistant gene, a blasticidin resistant gene, a dihydrofolate reductase (dhfr) gene, and a glutamine synthetase gene. In some embodiments, the vector comprises two or more antibiotic resistance genes.

Promoters suitable for use in the vectors depend on the host into which the vector is being introduced. Exemplary bacterial promoters include, but are not limited to, lac, T5, T7, tac, phage λPL, lpp, trp, penP, and SPO1 promoters. Exemplary yeast promoters include, but are not limited to, PHO5, PGK, GAP, ADH1, SUC2, GAL4, GAL1, Mfa, and AOX1 promoters. Exemplary mammalian promoters include, but are not limited to, CMV, SV40 (early or late), LTR from retrovirus, HSV-TK, and metallothionein promoters. Exemplary insect promoters include, but are not limited to, polyhedral and P10 promoters. Exemplary plant promoters include, but are not limited to, 35S and rice actin gene promoters. The promoters preferably lack a translation initiation sequence.

In some embodiments, the vectors comprise an enhancer. Suitable enhancers include, but are not limited to, SV40 enhancer, adenovirus enhancer, and cytomegalovirus early enhancer.

In some embodiments, the vectors comprise a ribosome binding site, a polyadenylation site (e.g., SV40 polyadenylation site), a splice donor and acceptor site (e.g., DNA sequence derived from SV40 splice site and the like), a transcription termination sequence, and a 5′ non-transcription sequence.

In one embodiment, the vector comprises the nucleic acid sequence set forth in SEQ ID NO: 1. This sequence corresponds to a vector with a cloning site immediately upstream of a lacZα gene, which lacks a translation initiation sequence. Accordingly, when used in blue-white screening in bacteria, cells harboring the empty vector (i.e., without an insert) would appear white, since lacZα is not expressed. Conversely, when a nucleic acid encoding a polypeptide, such as an antibody heavy chain variable region, that includes a native translation initiation sequence, is cloned upstream of the LacZα gene and in-frame with the reporter gene, a heavy chain variable region-LacZα fusion protein will be expressed, resulting in blue colonies being formed when bacteria are transformed with the vector. In one embodiment, the vector consists essentially of the nucleic acid sequence set forth in SEQ ID NO: 1. In some embodiments, the vector may comprise a nucleic acid sequence that is at least 95%, at least 96%, at least 97%, at least 98%, or at least 99% identical to SEQ ID NO: 1. In some embodiments, the vector may comprise a nucleic acid sequence that is at least 95% identical to SEQ ID NO: 1. In some embodiments, the vector may comprise a nucleic acid sequence that is at least 98% identical to SEQ ID NO: 1.

The platform concept described herein can be applied to any suitable vector. For example, existing (e.g., commercial) vectors, such as pUC19, can be modified such that a reporter gene that lacks a translation initiation sequence (e.g., lacZα without translation initiation sequence) is cloned using gene-specific primers and introduced into the vector cloning site (e.g., multiple cloning site), and two unique cloning sites are added (if not already present in the vector) immediately upstream of the reporter gene to allow for cloning of a nucleic acid encoding a polypeptide (e.g., antibody variable region sequence), such that the native translation initiation sequence of the nucleic acid encoding a polypeptide drives expression of the polypeptide and reporter gene (e.g., as a fusion protein).

Accordingly, in another aspect, provided herein is a method of preparing a modified vector comprising:

(a) cloning a reporter gene using gene specific primers such that the reporter gene lacks a translation initiation sequence,

(b) introducing the reporter gene into the cloning site of the vector, and

(c) if not already present, introducing unique cloning sites in the vector immediately upstream of the nucleotide sequence encoding the reporter gene,

wherein the reporter gene is not expressed or expressed at very low level when the vector is introduced into a host cell in the absence of an upstream nucleic acid encoding a polypeptide.

In some embodiments, if a restriction site for cloning the nucleic acid encoding the polypeptide is not unique in the vector (e.g., if it is also present in the sequence of the reporter gene), site-directed mutagenesis can be used to remove the restriction site in the reporter gene sequence without altering the encoded amino acid.

The vectors can be introduced into host cells to generate transformants using standard methods known in the art.

Libraries of polypeptides can be obtained using standard methods known in the art. For example, libraries of antibody variable regions can be prepared from nucleic acids obtained from antibody-producing cells by amplifying variable region sequences with PCR using specifically designed primers which include 15-bp adapter sequences that are complementary to the ends of the linearized vector. The primers may be designed such that the polypeptides are cloned into the vector in-frame with the reporter gene. Reverse primers are gene specific and directed to conserved sequences located after the 3′ terminus of the variable region. In some embodiments, the gene specific primers incorporate adapter sequence to the vector to facilitate directional cloning.

Suitable host cells include, but are not limited to, prokaryotic cells (e.g., bacterial cells), yeast cells, plant cells, and mammalian cells (e.g., animal cells). Suitable bacterial cells for introducing the vector into and expressing include, but are not limited to, E. coli, Bacillus subtillis, Salmonella, Pseudomonas, Streptomyces, and Staphylococcus. Suitable yeast cells include, but are not limited to, Kluyveromyces Lactis, Saccharomyces cerevisiae, Schizosaccharomyces pombe, and Pichia pastoris. Suitable mammalian cells include, but are not limited to, 293 cells, COS-7 cells, HeLa cells, CHO cells, NSO cells, AtT-20 cells, GH3 cells, MtT cells, MIN6 cells, Vero cells, C127 cells, CHO cells, BHK cells, and 3T3 cells.

Also provided herein are methods of screening for antibody variable regions. In one embodiment, the method comprises:
a) amplifying one or more antibody variable regions using primers (e.g., universal 5′ RACE-PCR primers for the forward primer, and gene-specific primer as the reverse primer);
b) cloning the amplified product into a vector described herein;
c) transforming the vector of step b) into a host cell; and
d) screening for cells that express the reporter gene,

wherein high levels of expression of the reporter gene is indicative of the cloning of a full length variable region, and low levels or no expression of the reporter gene is indicative of the cloning of a non-full length variable region.

In another aspect, provided herein is a method of screening for antigen binding molecules comprising:
a) amplifying a nucleic acid encoding an antigen binding molecule using gene specific primers;
b) cloning the amplified nucleic acid into the vector of any one of claims 1-21, wherein the amplified nucleic acid is inserted in-frame with the reporter gene in the vector;
c) transforming the vector resulting from step b) into host cells; and
d) screening for cells that express the protein encoded by the reporter gene,

wherein expression of the protein encoded by the reporter gene is indicative of presence of a native translation initiation sequence in the amplified nucleic acid encoding the antigen binding molecule.

In another aspect, provided herein is a method of screening for antigen binding molecules comprising:
a) amplifying a nucleic acid encoding an antigen binding molecule using gene specific primers;
b) cloning the amplified nucleic acid into a vector, wherein the vector comprises

(i) a cloning site for directional cloning of a nucleic acid, and

(ii) a reporter gene lacking translation initiation sequence,

wherein the cloning site is upstream of the reporter gene, and

wherein the amplified nucleic acid is inserted into the cloning site in-frame with the reporter gene;
c) transforming the vector containing the amplified nucleic acid into host cells; and
d) screening for cells that express the protein encoded by the reporter gene.

In some embodiments, the antigen binding molecule may be a variable region of an antibody. In some embodiments, the variable region of the antibody is a heavy chain variable region. In some embodiments, the variable region of the antibody is a light chain variable region. In some embodiments, the antigen binding molecule is a variable region of a T cell receptor (TCR). In some embodiments, the antigen binding molecule is a variable region of a TCR alpha (α), beta (β), gamma (γ), or delta (δ) chain

In some embodiments of the screening method, the amplified product is cloned into the vector by directional cloning, e.g., using IN-FUSION® cloning.

In some embodiments of the screening method, the host cell is selected from the group consisting of a bacterial cell, a yeast cell, an insect cell, a plant cell, and a mammalian cell.

In some embodiments, the reporter gene is lacZα or an enzymatically active variant thereof. In some embodiments, cells that are blue are selected. In some embodiments, cells that are dark blue are selected. In certain embodiments, cells that are dark blue are considered to contain a cloned full length variable region.

In some embodiments, the screening method further comprises the step of selecting a cell or cells that express the reporter gene and sequencing the variable region in the vector.

In some embodiments, the screening method is performed in a high throughput format, e.g., using 96-well plates.

In some embodiments, the screening method further comprises the step of screening for antibodies having high affinity for the antigen of interest using standard methods known in the art.

Also provided herein are kits comprising the vector and instructions for use. In some embodiments, the vector is linearized. In some embodiments, the vector is linearized using two restriction sites in the cloning site that are unique in the vector (i.e., there is only one of each restriction site in the vector). In some embodiments, the kits comprise host cells. In some embodiments, the kits comprise auxiliary components (e.g., buffers, restriction enzymes, primers, libraries, reporter gene substrates).

The present invention is further illustrated by the following examples which should not be construed as further limiting. The contents of Sequence Listing, figures and all references, patents and published patent applications cited throughout this application are expressly incorporated herein by reference.

### EXAMPLES

Commercially available reagents referred to in the Examples below were used according to manufacturer's instructions unless otherwise indicated. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

### Example 1: Generation of a Vector that Relies on Native Translation Initiation Elements for Gene Expression

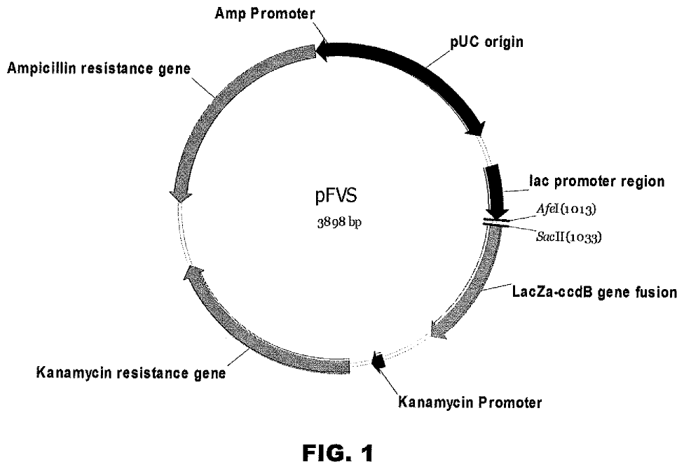

This Example describes the generation of the pFVS vector, which relies on native translation initiation elements of cloned PCR fragments to drive the expression of a reporter gene. FIG. 1 provides a schematic of the pFVS vector, which is a modified version of the pCR4.TOPO vector.

Briefly, the pCR4.TOPO vector was digested with PciI and BsiWI to provide the backbone vector (˜3331 bps). A DNA fragment (596 bp; SEQ ID NO: 2) was synthesized, containing overlapping sequences (underlined in Table 3) at both ends with the digested backbone vector. An AfeI site, a SacII site, and a modified LacZα gene (no codon for start methionine) were designed and introduced in the synthesized DNA fragments. pFVS was generated by in fusion cloning this synthesized DNA into the backbone vector. The sequence of pFVS is provided in SEQ ID NO: 1. Components of the vector are as follows (the numbers in parentheses indicate positions in the vector relative to the first base pair of pUC origin): pUC origin (1-674 bp), LacZ-ccdB gene fusion (1033-1548 bp), Lac promoter region (799-1012 bp), Kanamycin resistance gene (1897-2691 bp), Ampicillin resistance gene (2941-3801 bp), AfeI site (1013 bp), SacII site (1033 bp).

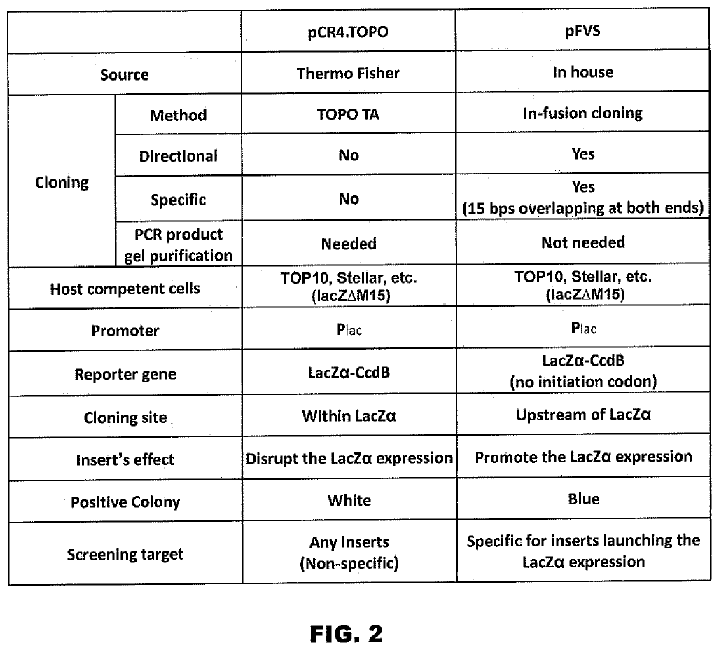

FIG. 2 provides a general overview of the features of the pFVS vector, as well as a comparison of these features with those of the pCR4.TOPO vector. The pFVS vector allows for in-fusion cloning, which provides for directional, efficient, seamless, and precise cloning, without the need to purify the PCR product (insert) prior to cloning. In contrast to the pCR4.TOPO vector, which includes a cloning site within the LacZα reporter gene, the cloning site of the pFVS vector is located upstream of the LacZα reporter gene. Since the LacZα reporter gene lacks an initiation codon in the pFVS vector, only inserts with a translation initiation codon drive LacZα expression. This allows for the efficient screening of blue colonies, which are driven by full-length inserts, as opposed to the screening of white colonies with the pCR4.TOPO vector. Additional benefits of the vector include convenience for sequencing analysis (due to directional cloning, sequencing data is analyzed only in one direction in contrast to data obtained from pCR4.TOPO cloning, which must be analyzed in both directions); fewer colonies to pick for sequencing studies, given the high probability of obtaining full length variable region sequences; and direct cloning of PCR fragments into the vector, allowing high throughput processing of samples (e.g., in 96-well plates).

### Example 2: Variable Region Screening Using pFVS Vector

This Example describes the proof of concept of the high efficiency of colony screening for antibody variable region sequences using the pFVS vector.

Briefly, total RNA was prepared from an anti-RANKL antibody producing hybridoma cell line, and first strand cDNA was synthesized with the Clontech SMARTer RACE (rapid amplification of cDNA ends) kit.

For pCR4.TOPO cloning, the variable region of the antibody heavy chain was amplified using the 5′-RACE PCR procedure with a rat-constant region specific reverse primer paired with the 5′ RACE universal primer mix. The resultant PCR product containing the rat VH was purified from an agarose gel by gel extraction with the NuceloSpin Gel and PCR clean up kit. The purified rVH was further cloned into the pCR4-TOPO vector following the TOPO TA cloning protocol.

For pFVS in fusion cloning, the variable region of the antibody heavy chain was amplified using the 5′-RACE PCR procedure with a modified rat-constant region specific reverse primer paired with the modified 5′ RACE universal primer mix. The primers were modified by adding 15-bp overlapping sequences with the ends of pFVS digested with AfeI and SacII restriction enzymes. The resultant PCR product containing the rat VH was purified from an agarose gel by gel extraction with the NuceloSpin Gel and PCR clean up kit. Both purified and non-purified PCR products were cloned into the pFVS vector following the In-Fusion HD Cloning Kit user manual.

The pCR4.TOPO cloning reaction and pFVS In-Fusion Cloning reaction were transformed into competent E. Coli cells (TOP10, Stellar, etc.) with a lacZΔM15 genomic background to allow blue/white color screening on LB agar plates containing 100 μg/ml carbenicillin, 0.1 mM IPTG and 60 μg/ml X-gal.

TempliPhi samples were prepared with 1) white colonies from pCR4.TOPO cloning, 2) dark blue colonies from pFVS cloning, and 3) light blue or white colonies from pFVS cloning. After DNA sequencing, the resultant DNA sequences were analyzed for in-frame rearrangements and other antibody characteristics.

As shown in Table 1, colony screening based on the pFVS vector was substantially more efficient than screening based on the pCR4.TOPO vector. Specifically, while all 4 dark blue colonies picked in the pFVS vector-based screening (with or without gel extraction purification of the insert) were positive for the anti-RANKL antibody VH insert, only 5 of 32 colonies picked were positive for the insert using the pCR4.TOPO vector. Moreover, colony screening based on the pFVS vector had very low false negative background, as demonstrated by only 1 hit among 12 white/light blue colonies generated from purified PCR product and no hits among 12 white/light blue colonies generated from non-purified PCR product. The resulting variable region cDNA sequence was used to predict the molecular weight for the whole heavy chain, which agreed with the molecular weight observed from Mass spec. data conducted on the antibody purified from the corresponding hybridoma supernatant.

TABLE 1

| | | | pFVS | | | |
|---|---|---|---|---|---|---|
| | pCR4.TOPO | | | | White or light blue | |
| | TA cloning | | Dark blue colonies | | colonies | |
| Insert | | colonies | | colonies | | colonies |
| RANKL rVH | hits | picked | hits | picked | hits | picked |
| PCR band | 5 | 32 | 4 | 4 | 1 | 12 |
| (gel extraction purified) | | | | | | |
| PCR reaction directly | NA | NA | 4 | 4 | 0 | 12 |

### Example 3: pFVS Improves the Frequency of Sequences

This Example compares the proportion of sequences obtained using the pCR4.TOPO vector compared to the pFVS vector. Briefly, gene-specific reverse primers were designed to amplify the heavy and light chain variable region sequences of antibodies from different species, including human, mouse, rat, and hamster. Several V region sequencing projects for hybridoma cell pellets from different species were included in this study. For the pCR4.TOPO vector, PCR amplified products were gel purified and subjected to TA cloning, followed by white colony screening and Sanger sequencing. For the pFVS vector, 5′RACE PCR was performed without subsequent gel purification, followed by in-fusion cloning and transformation. Dark blue colonies were picked for Sanger sequencing.

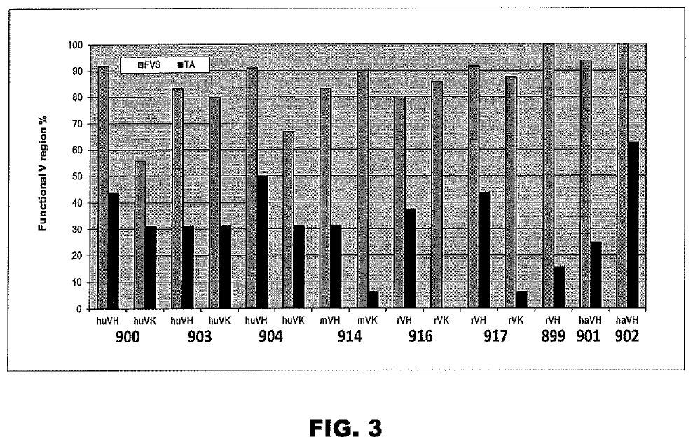

As shown in FIG. 3, in all 9 variable region sequencing projects compared in this study, 16 colonies were picked for each V region sequenced with pCR4.TOPO cloning and 5 to 16 colonies were picked for each V region sequenced with pFVS cloning. In each case, the proportion of full length heavy and light chain variable regions obtained using the pFVS vector was substantially greater than that obtained using the pCR4.TOPO vector. On the basis of these data, the average probability and its standard deviation, as well as the probability of false positives, were calculated (Table 2). The probability of the false negatives also was calculated from the VH region sequencing study of the anti-RANKL antibody described in Example 2.

Table 2 summarizes the probability of false positives and false negatives with the pFVS and pCR4.TOPO approaches.

TABLE 2

| Probability | pFVS | pCR4.TOPO |
|---|---|---|
| Average | 85.35% | 29.79% |
| Standard deviation | 11.76% | 17.23% |
| False positives | 14.65% | 70.21% |
| False negatives | 4.2% | Unknown |

From the sequencing data analyses, it was observed that false positives (dark blue colony but no full length V region) from the pFVS vector could result from 5′ end truncated V regions with an internal methionine, V regions having an frame-shifted internal methionine reading through and in-frame with the LacZα reporter, alternative start codons, or different code usage in E. coli and B cells. False negatives (white or light blue colony with full length V region) likely derived from the about 5% error rate of the in-fusion cloning process. Nonetheless, as can be seen from Table 2, the pFVS vector serves as a more efficient and precise tool for screening full-length polypeptide inserts, such as antibody variable region sequences.

### Example 4: Application to Other Vectors

The platform strategy described in the preceding Examples can be used with a different starting vector, for example, a vector having GFP as a reporter gene. GFP exhibits bright green fluorescence when exposed to light in the blue to ultraviolet and requires no substrate or cofactors to fluoresce. The start codon for the first methionine of GFP is deleted and the remaining sequence is designed in-frame with cloned DNA amplified by RACE PCR as described in the preceding Examples. For such vectors, only cloned DNA fragments containing full-length antibody variable regions initiate the translation and read through of the GFP gene in frame, resulting in colonies that fluoresce under blue light or ultraviolet light. These positive colonies can be picked for sequencing studies.

TABLE 3
Summary of sequences

| SEQ | | |
|---|---|---|
| ID | Description | Sequence |
| 1 | pFVS vector | ccccgtagaaaagatcaaaggatcttcttgagatcctttttttctgcgcgtaatctgctgcttgcaaacaaaaaaac |
| | pUC origin | caccgctaccagcggtggtttgtttgccggatcaagagctaccaactctttttccgaaggtaactggcttcagca |
| | (1-674 bp); | gagcgcagataccaaatactgtccttctagtgtagccgtagttaggccaccacttcaagaactctgtagcaccg |
| | LacZa-ccdB | cctacatacctcgctctgctaatcctgttaccagtggctgctgccagtggcgataagtcgtgtcttaccgggttgg |
| | gene fusion | actcaagacgatagttaccggataaggcgcagcggtcgggctgaacggggggttcgtgcacacagcccagc |
| | (1033-1548 | ttggagcgaacgacctacaccgaactgagatacctacagcgtgagctatgagaaagcgccacgcttcccgaa |
| | bp); | gggagaaaggcggacaggtatccggtaagcggcagggtcggaacaggagagcgcacgagggagcttcc |
| | Lac promoter | agggggaaacgcctggtatctttatagtcctgtcgggtttccgccacctctgacttgagcgtcgatttttgtgatgct |
| | region | cgtcaggggggcggagcctatggaaaaacgccagcaacgcggcctttttacggttcctggccttttgctggcc |
| | (799-1012 | ttttgctcacatgttctttcctgcgttatcccctgattctgtggataaccgtattaccgcctttgagtgagctgatacc |
| | bp); | gctcgccgcagccgaacgaccgagcgcagcgagtcagtgagcgaggaagcggaagagcgcccaatacg |
| | Kanamycin | caaaccgcctctccccgcgcgttggccgattcattaatgcagctggcacgacaggtttcccgactggaaagcg |
| | resistance | ggcagtgagcgcaacgcaattaatgtgagttagctcactcattaggcaccccaggctttacactttatgcttccg |
| | gene (1897- | gctcgtatgttgtgtggaattgtgagcggataacaatttcacacaggaaacagcgctttttagctttaaaccgcgg |
| | 2691 bp); | ggatcccaaacttcttctggaggtaccgcatgcgatttcgagctctcccggcaattcactggccgtcgttttacaa |
| | Ampicillin | cgtcgtgactgggaaaaccctggcgttacccaacttaatcgccttgcagcacatccccctttcgccagctggcg |
| | resistance | taatagcgaagaggcccgcaccgatcgcccttcccaacagttgcgcagcctatacgtacggcagtttaaggttt |
| | gene (2941- | acacctataaaagagagagccgttatcgtctgtttgtggatgtacagagtgatattattgacacgccggggcgac |
| | 3801 bp); | ggatggtgatccccctggccagtgcacgtctgctgtcagataaagtctcccgtgaactttacccggtggtgcata |
| | AfeI site | tcggggatgaaagctggcgcatgatgaccaccgatatggccagtgtgccggtctccgttatcggggaagaag |
| | (1013 bp); | tggctgatctcagccaccgcgaaaatgacatcaaaaacgccattaacctgatgttctggggaatataaatgtca |
| | SacII site | ggcatgagattatcaaaaaggatcttcacctagatccttttcacgtagaaagccagtccgcagaaacggtgctg |
| | (1033 bp) | accccggatgaatgtcagctactgggctatctggacaagggaaaacgcaagcgcaaagagaaagcaggtag |
| | | cttgcagtgggcttacatggcgatagctagactgggcggttttatggacagcaagcgaaccggaattgccagc |
| | | tggggcgccctctggtaaggttgggaagccctgcaaagtaaactggatggctttctcgccgccaaggatctga |
| | | tggcgcaggggatcaagctctgatcaagagacaggatgaggatcgtttcgcatgattgaacaagatggattgc |
| | | acgcaggttctccggccgcttgggtggagaggctattcggctatgactgggcacaacagacaatcggctgctc |
| | | tgatgccgccgtgttccggctgtcagcgcaggggcgcccggttctttttgtcaagaccgacctgtccggtgccc |
| | | tgaatgaactgcaagacgaggcagcgcggctatcgtggctggccacgacgggcgttccttgcgcagctgtgc |
| | | tcgacgttgtcactgaagcgggaagggactggctgctattgggcgaagtgccggggcaggatctcctgtcatc |
| | | tcaccttgctcctgccgagaaagtatccatcatggctgatgcaatgcggcggctgcatacgcttgatccggcta |
| | | cctgcccattcgaccaccaagcgaaacatcgcatcgagcgagcacgtactcggatggaagccggtcttgtcg |
| | | atcaggatgatctggacgaagagcatcaggggctcgcgccagccgaactgttcgccaggctcaaggcgagc |
| | | atgcccgacggcgaggatctcgtcgtgacccatggcgatgcctgcttgccgaatatcatggtggaaaatggcc |
| | | gcttttctggattcatcgactgtggccggctgggtgtggcggaccgctatcaggacatagcgttggctacccgt |
| | | gatattgctgaagagcttggcggcgaatgggctgaccgcttcctcgtgctttacggtatcgccgctcccgattcg |
| | | cagcgcatcgccttctatcgccttcttgacgagttcttctgaattattaacgcttacaatttcctgatgcggtattttct |
| | | ccttacgcatctgtgcggtatttcacaccgcatacaggtggcacttttggggaaatgtgcgcggaacccctattt |
| | | gtttatttttctaaatacattcaaatatgtatccgctcatgagacaataaccctgataaatgcttcaataatattgaaaa |
| | | aggaagagtatgagtattcaacatttccgtgtcgcccttattcccttttttgcggcattttgccttcctgtttttgctca |
| | | cccagaaacgctggtgaaagtaaaagatgctgaagatcagttgggtgcacgagtgggttacatcgaactggat |
| | | ctcaacagcggtaagatccttgagagttttcgccccgaagaacgttttccaatgatgagcacttttaaagttctgct |
| | | atgtggcgcggtattatcccgtattgacgccgggcaagagcaactcggtcgccgcatacactattctcagaatg |
| | | acttggttgagtactcaccagtcacagaaaagcatcttacggatggcatgacagtaagagaattatgcagtgct |
| | | gccataaccatgagtgataacactgcggccaacttacttctgacaacgatcggaggaccgaaggagctaacc |
| | | gcttttttgcacaacatgggggatcatgtaactcgccttgatcgttgggaaccggagctgaatgaagccatacca |
| | | aacgacgagcgtgacaccacgatgcctgtagcaatggcaacaacgttgcgcaaactattaactggcgaacta |
| | | cttactctagcttcccggcaacaattaatagactggatggaggcggataaagttgcaggaccacttctgcgctc |
| | | ggcccttccggctggctggtttattgctgataaatctggagccggtgagcgtgggtctcgcggtatcattgcagc |
| | | actggggccagatggtaagccctcccgtatcgtagttatctacacgacggggagtcaggcaactatggatgaa |
| | | cgaaatagacagatcgctgagataggtgcctcactgattaagcattggtaactgtcagaccaagtttactcatata |
| | | tactttagattgatttaaaacttcatttttaatttaaaaggatctaggtgaagatcctttttgataatctcatgaccaaaa |
| | | tcccttaacgtgagttttcgttccactgagcgtcaga |
| 2 | Synthesized | gccttttgctcacatgttctttcctgcgttatcccctgattctgtggataaccgtattaccgcctttgagtgagctgat |
| | DNA | accgctcgccgcagccgaacgaccgagcgcagcgagtcagtgagcgaggaagcggaagagcgcccaata |
| | fragment | cgcaaaccgcctctccccgcgcgttggccgattcattaatgcagctggcacgacaggtttcccgactggaaag |
| | | cgggcagtgagcgcaacgcaattaatgtgagttagctcactcattaggcaccccaggctttacactttatgcttc |
| | | cggctcgtatgttgtgtggaattgtgagcggataacaatttcacacaggaaacagcgctttttagctttaaaccgc |
| | | ggggatcccaaacttcttctggaggtaccgcatgcgatttcgagctctcccggcaattcactggccgtcgttttac |
| | | aacgtcgtgactgggaaaaccctggcgttacccaacttaatcgccttgcagcacatccccctttcgccagctgg |
| | | cgtaatagcgaagaggcccgcaccgatcgcccttcccaacagttgcgcagcctatacgtacggcagtttaaggt |

### EQUIVALENTS

Those skilled in the art will recognize, or be able to ascertain using no more than routine experimentation, many equivalents of the specific embodiments disclosed herein. Such equivalents are intended to be encompassed by the following claims.

1. A vector comprising (i) a cloning site for directional cloning of a nucleic acid encoding an antigen binding molecule comprising a native translation initiation sequence, and (ii) a reporter gene lacking translation initiation sequence,
wherein the cloning site is upstream of the reporter gene, and when the nucleic acid encoding the antigen binding molecule is cloned into the cloning site, the expression of the reporter gene is driven by the native translation initiation sequence in the nucleic acid encoding the antigen binding molecule.

2. The vector of claim 1, wherein the antigen binding molecule is a variable region of an antibody or a T cell receptor (TCR).

3. The vector of claim 1 wherein the vector comprises a promoter for expression of the antigen binding molecule.

4. The vector of claim 1, wherein the promoter is a prokaryotic promoter.

5.-9. (canceled)

10. The vector of claim 1, wherein the reporter gene comprises a gene encoding an enzyme, a chromogenic protein, a fluorescent protein, or a toxic gene.

11.-16. (canceled)

17. The vector of claim 1, wherein the antibody binding molecule is a heavy chain variable region of an antibody.

18. The vector of claim 1, wherein the antibody binding molecule is a light chain variable region of an antibody.

19. A vector comprising a nucleotide sequence that is at least 95% identical to SEQ ID NO: 1.

20. The vector of claim 19, wherein the nucleotide sequence is at least 98% identical to SEQ ID NO: 1.

21. (canceled)

22. A kit comprising the vector of claim 1, and instructions for use.

23.-24. (canceled)

25. A method of screening for antigen binding molecules comprising:
a) amplifying a nucleic acid encoding an antigen binding molecule using gene specific primers;
b) cloning the amplified nucleic acid into a vector, wherein the vector comprises
(i) a cloning site for directional cloning of a nucleic acid, and
(ii) a reporter gene lacking translation initiation sequence,
wherein the cloning site is upstream of the reporter gene, and
wherein the amplified nucleic acid is inserted into the cloning site in-frame with the reporter gene;
c) transforming the vector containing the amplified nucleic acid into host cells; and
d) screening for cells that express the protein encoded by the reporter gene.

26. The method of claim 25, wherein the antigen binding molecule is a variable region of an antibody.

27. The method of claim 26, wherein the variable region of the antibody is a heavy chain variable region.

28. The method of claim 26, wherein the variable region of the antibody is a light chain variable region.

29. The method of claim 25, wherein the antigen binding molecule is a variable region of a T cell receptor (TCR).

30. (canceled)

31. The method of claim 25, wherein the vector comprises a nucleotide sequence that is at least 95% identical to SEQ ID NO: 1.

32. The method of claim 25, wherein the vector comprises a nucleotide sequence that is at least 98% identical to SEQ ID NO: 1.

33.-35. (canceled)

36. The method of claim 25, wherein the vector comprises a promoter for expression of the antigen binding molecule.

37.-42. (canceled)

43. The method of claim 25, wherein the reporter gene comprises a gene encoding an enzyme, a chromogenic protein, a fluorescent protein, or a toxic gene.

44.-54. (canceled)

55. The method of claim 25, further comprising the step of sequencing the amplified nucleic acid encoding the antigen binding molecule in the cells that express the protein encoded by the reporter gene.
